(19) United States
(12) Patent Application Publication
(10) Pub. No.: US 2010/0286507 A1
(43) Pub. Date: Nov. 11, 2010

(54) DETERMINING THE POSITION OF A NEEDLE

(76) Inventors: Katja Paassilta, Kangasala (FI); Marjo-Riitta Suuronen, Pirkkala (FI); Jari Hyttinen, Tampere (FI)

Correspondence Address: BIRCH STEWART KOLASCH & BIRCH, PO BOX 747, FALLS CHURCH, VA, 22040-0747, US

(21) Appl. No.: 12/811,152

(22) PCT Filed: Dec. 29, 2008
(86) PCT No.: PCT/FI08/50788

§ 371 Date: Jun. 29, 2010

(30) Foreign Application Priority Data
Dec. 31, 2007 (FI) 20075978

(51) Int. Cl. A61B 5/05

(52) U.S. Cl. 600/424

(57) ABSTRACT

An arrangement for determining the position of a needle in an organ system and a method utilizing the arrangement in cosmetic treatment or cosmetic surgery. The arrangement (1) comprises a needle (2) with a distal tip (3), a measurement circuit (4) measuring the bioimpedance of tissues, a detection means (10) connected to the measurement circuit measuring bioimpedance and arranged to detect the progress of the distal tip (3) of the needle at a real-time basis from the bioimpedance measured from tissues. The arrangement (1) comprises at least two electrodes (16, 18, 21, 22) arranged at the distal tip (3) of the needle and connected to the measurement circuit (4). All the electrodes (16, 18, 21, 22) measuring impedance and needed for monitoring the progress of the distal tip (3) of the needle are arranged to the distal tip (3) of the needle.

### BACKGROUND OF THE INVENTION

The invention relates to an arrangement for determining the position of a needle in an organ system, the arrangement comprising a needle having a distal tip, a measurement circuit measuring the bioimpedance of tissues, a detection means, the detection means being connected to the measurement circuit measuring the bioimpedance and arranged to detect the progress of the distal tip of the needle in the tissue on a real-time basis from the bioimpedance measured from the tissues.

The invention further relates to a method for a cosmetic treatment or cosmetic surgery.

In a number of therapeutic, surgical, diagnostic and cosmetic measures drugs or substances, such as tracers, relating to the treatment are administered into the patient's organ system. These substances are usually in a liquid or gaseous form and delivered to the tissue through a hollow needle. Medical procedures in which substances are removed from the body through a hollow needle or biopsies are taken from a tissue with a needle are also known.

In all the above-mentioned measures it is of an utmost importance to be able to take the tip of the needle to the correct tissue and to know when the tip is in the correct tissue. For example, local anesthesia of the spinal nerves, i.e. spinal or epidural anesthesia may cause severe complications, such as unconsciousness or even death, if the anesthetic is administered to a wrong tissue.

At the moment prior art knows mechanical instruments with which a physician obtains information on the position of a needle in a tissue. However, such instruments are not totally reliable. The physician may also rely on his/her knowledge of anatomy. Nevertheless, the human anatomic structure varies a lot from one individual to another. Often the physician is forced to trust on “feel” alone.

Prior art also knows systems for determining needle position on the basis of bioimpedance measurements, for example. One of these is disclosed in publication WO 2007/075 091. However, the complicated structure of the system and shortcomings due to the electrodes to be attached to the skin cause problems relating to how promptly the system is available for use and its usability.

### BRIEF DISCLOSURE OF THE INVENTION

It is an object of the invention to provide a novel and improved system and method.

The arrangement of the invention is characterized in that it comprises at least two electrodes arranged to the distal tip of the needle, the electrodes being coupled to the measurement circuit, and that all electrodes measuring bioimpedance and needed for monitoring the progress of the distal tip of the needle are arranged to the distal tip of the needle.

The method of the invention is characterized by comprising the following steps of: arranging for use a needle with a distal tip, in which needle all electrodes measuring bioimpedance and needed for monitoring the progress of the distal tip of the needle are arranged to the distal tip, arranging for use a measurement circuit measuring the bioimpedance of tissues, and a detection means that is connected to the measurement circuit measuring bioimpedance; arranging the detection means to detect the progress of the distal tip of the needle in the tissue on a real-time basis from the bioimpedance measured from the tissues; connecting the electrodes to the measurement circuit; inserting the needle into the patient's tissue, and measuring the bioimpedance of the tissue.

An advantage of the invention is that since all electrodes needed in the measurement of bioimpedance are arranged to the needle, the arrangement is extremely easy and rapid to use. A further advantage is that since the electrodes are arranged to the distal tip, it is possible to measure tissues that are extremely close to the tip.

According to an embodiment of the invention the arrangement comprises means for detecting a force that resists the progress of the needle in the tissue. These means may comprise a power sensor or a pressure sensor, for example. An advantage of this is the possibility to detect a hard/solid tissue located in front of the distal tip of the needle and thus to avoid the needle becoming broken.

According to another embodiment of the invention the arrangement comprises a master station to be arranged to the patient's skin or mucosa, the master station comprising a frame structure attachable to the patient and at least one power member arranged to transfer the needle in relation to the frame structure and, further, sounding means arranged to detect the patient's tissue through his/her skin or mucosa.

An advantage of the master station is that it allows the needle to be correctly positioned at a correct angle to the skin or the mucosa with a significantly greater precision than by manual positioning. Also when the needle is inserted into the tissue, movements assisted by the power member are significantly more accurate than movements of a hand. After the master station has been fastened, the system may advantageously operate independently, the entire operation taking place automatically at the press of a button.

According to an embodiment the arrangement comprises memory means for storing an anatomy model produced on the basis of anatomy data and means for adjusting the bioimpedance data measured by the electrodes to the model in such a way that the position of the distal tip of the needle in relation to the anatomy model is presentable on a display device, such as a screen.

An advantage is that the position of the distal tip of the needle in relation to the tissues is presentable to the personnel using the system in an illustrative and clear manner so that any special skills for interpreting the measurement results are not necessary for determining the position of the needle.

### BRIEF DISCLOSURE OF THE FIGURES

Some embodiments of the invention will be disclosed in greater detail in the accompanying drawings, in which

FIG. 1 is a schematic diagram of an arrangement of the invention;

FIG. 2 is a schematic diagram of a processing unit belonging to the arrangement of the invention;

FIG. 3 is a schematic view of a needle belonging to the arrangement of the invention;

FIG. 4 is a schematic view of a second needle belonging to the arrangement of the invention;

FIG. 5 is a schematic view of a third needle belonging to the arrangement of the invention;

FIG. 6 is a schematic view of a fourth needle belonging to the arrangement of the invention;

FIGS. 7a and 7b are schematic views of a master station of the arrangement of the invention in a first and a second position;

FIGS. 8a to 8d are schematic views of an arrangement of the invention in epidural anesthesia;

FIG. 9 shows a typical bioimpedance curve modelled with the arrangement of the invention; and

FIG. 10 shows the conductivity ratio of some tissue types as a function of measurement frequency.

For the sake of clarity, some embodiments of the invention have been simplified in the figures. Like parts are referred to with like reference numerals.

### DETAILED DESCRIPTION OF THE INVENTION

FIG. 1 is a schematic diagram of an arrangement of the invention. The arrangement 1 employs bioimpedance measurement and, further, a power sensor for detecting tissues and for distinguishing them from one another. Compared with previous solutions, the arrangement offers a solution that is easier to use for example for preventing accidental injections to a wrong tissue. The parts and components of the arrangement may be combined with a syringe, catheter, sampling device or similar device structures comprising a needle to be inserted into the organ system in such a way that the arrangement will not impair or essentially change the practices followed in the use of such devices.

In the following some embodiments of the invention and their use are disclosed in connection with epidural anesthesia, i.e. anesthesia of the spinal nerves. However, it is evident that the arrangement may be used not only in epidural anesthesia but also in numerous other forms of anesthesia, for example in therapeutic, surgical, diagnostic and cosmetic procedures.

The arrangement 1 comprises a needle 2 with a distal tip 3. The distal tip is the end of the needle that penetrates the tissue. The needle 2 has been arranged as a part of a syringe 9, which comprises not only the needle but also a container, in a manner known per se, into which the substance to be injected is arranged, and a piston for forcing the substance to be injected through the needle 2 into the organ system. In this embodiment the needle 2 is hollow and comprises a conduit for delivering the substance to be injected into the body. The substance to be injected may be a liquid or gaseous substance known per se.

It should be noted at this point that in some other embodiments the arrangement comprises a needle and other necessary equipment for removing substances from the organ system or for taking samples from tissues. It should also be noted that in some further embodiments the needle 2 is not hollow.

The arrangement 1 further comprises a measurement circuit 4 with a processing unit 5 based on a microprocessor for measuring bioimpedance. The processing unit 5 may be integrated into the syringe 9, catheter, sampling device or similar device comprising a needle monitored by the arrangement 1, or it may be separately arranged, to a PC for example, from the device comprising the needle to be monitored with the arrangement 1.

The needle 2 comprises electrodes 6 whose number here is four. The electrodes 6 are arranged to the distal tip 3 of the needle or to the immediate vicinity thereof. The electrodes 6 are connected to the measurement circuit 4 and coupled with wires 7 to the processing unit 5. The wires 7 may be integrated into the needle 2 and/or the syringe 9, similarly as the entire measurement circuit 4 and its processing units 5.

In FIG. 1 the illustration has been facilitated by depicting the wires 7 in the form of separate wires drawn from the distal tip 3 of the needle 2 directly to the processing unit 5. In reality the wires 7 are arranged to run along the structure of the needle 2 from an electrode to the rest of the structure of the syringe 9, and further through suitable connectors and wires to the processing unit 5. The wires 7 are shielded within the structure of the syringe 9 and do not impair the normal use of the syringe 9.

It is also possible to use wireless data transfer from the syringe 9 to the processing unit 5 arranged separately from it, whereby wires and cables impairing to some extent the handling of the syringe 9 are not needed in the data transfer.

All electrodes 6 measuring bioimpedance and detecting the position of the needle tip are arranged to the needle 2, actually as close to the distal tip 3 of the needle as possible, which preferably allows the bioimpedance of the tissue in front of the distal tip 3 of the needle to be measured.

The more electrodes 6 arranged to the needle, the more accurately the bioimpedance measurement may be carried out. With at least two electrodes 6 in the needle, the electrodes take care of both supplying the measurement current and measuring the impedance, and the measurement as a whole may be integrated into the needle and the device comprising it. The number of the electrodes may be two, three, four, five, etc. An advantage of a system of four electrodes is that it reduces error potentials caused by electrode impedances. When four electrodes are used, the measurement preferably takes place in the form of what is known as a four-wire measurement.

The arrangement 1 further comprises a power sensor or a pressure sensor 13. This is arranged to detect the magnitude of the force resisting the advancement of the needle 2 in the tissue. The magnitude of the force is affected, among other things, by the type and quality of the tissues around needle. The sensor 13 allows advance information to be gained on the tissue into which the needle 2 is about to penetrate. For example, if the needle 2 is about to hit a bone tissue, advance information about this is received through the pressure sensor 13. In that case the position of the needle 2 may be changed before it runs the risk of being broken against the hard bone tissue. Naturally this operation may be implemented also by using information gained through bioimpedance measurements or ultrasound, for example.

The measurement circuit further includes a voltage or power source 8 connected to the measurement circuit and supplying alternating voltage or alternating current to the measurement circuit and further to appropriate electrodes 6.

The processing unit 5 supplies measurement results obtained on the basis of the measurement results from the bioimpedance measurements and the pressure sensor 13 to the detection means 10. The detection means 10 is typically a display device, such as a crt or lcd display. The detection means 10 may also be a device producing a sound signal and indicating for example by changes in the frequency and/or volume of the sound that the needle is approaching its target. The detection means 10 may also be a sound-producing speech synthesizer producing spoken information on the movements and position of the needle.

The detection means 10 informs the position of the needle in the patient's organs system preferably on a continuous real-time basis from the moment when the distal tip 3 of the needle is inserted into the patient's skin or mucosa. The measurement results provided by the detection means 10 may be direct measurement results, for example, i.e. bioimpedance values provided by the electrodes 6 and measurement results supplied by the power sensor 13. Most preferably the detection means 10 is a display that enables to display a modelled image (a cross-section on a suitable plane) of the patient's anatomy and the real-time movement of the needle 2, or at least the distal tip 3 of the needle, in the modelled organ system of the patient. Thus the position of the needle 2 in the organ system can be displayed as clearly and illustratively as possible. The detection means 10 may also be a device giving a sound signal, or a signal light.

The arrangement further includes a memory means 11 for storing data in a digital format. The memory means 11 may be a hard disk, CD or DVD, flash memory or the like, known per se. The memory means 11 may also be connected to the processing unit 5 through an information network, in which case the memory means does not need to be physically in the same space with the processing unit 5 or the patient. However, it should be noted that the memory means 11 is not an indispensable part of the arrangement.

A computational model of the patient's anatomy may be stored in the memory means 11. The model may be produced either on the basis of information measured from the patient's body or commonly known anatomical information specific to a species—such as the human species—obtained by measuring the anatomy of a plural number of individuals representing the species, for example. The structure of the model may be produced on the basis of magnetic resonance images or other three-dimensional image material. It is also possible to produce the model on the basis of ultrasound images or conductivity values, i.e. bioimpedance. However, it should be noted that the modelling is not necessary, although it renders the operation and the use of the arrangement more precise.

In addition to the detection means 10, the arrangement 1 may comprise a signalling device 12. This may produce a visible and/or audible signal when the distal tip 3 of the needle reaches a tissue of a specific type. The signalling device 12 may comprise for example a signal light, sign or symbol on the display serving as the detection means 10, a sound-producing means, such as a loud speaker, and the signal to be provided by it may consist of spoken communication or a simple sound signal.

The arrangement may also include a dosage detector 14 measuring, in a manner known per se, the amount of the substance administered into the organ system or the amount of substance taken from the organ system. The detection may be based on detecting the movement of the piston in the syringe, for example. The detector 14 may be provided with automation that stops the dosage or the retrieval of the substance when a specific threshold value is reached.

Examples of using the arrangement in anesthesia include the following: the operator searches for the correct spot on the surface of the patient's skin or mucosa on the basis of his/her knowledge of anatomy and by palpating the area. Next, s/he points the needle 2 to the correct angle and manually pushes the needle 2 to the target through the tissues. When the arrangement 1 informs that the distal tip 3 of the needle has reached the desired position in the tissue, the operator injects a necessary amount of the anesthetic by pushing the piston of the syringe.

The arrangement enables to prevent the anesthetic from being injected to a wrong location. At the same time it reduces the risk of consequences caused to the patient, which in the worst case may be unconsciousness and death.

The use of the arrangement does not necessitate continuous presence of an experienced physician. A nurse, for example, may prepare the anesthesia operation to a fairly advanced state independently. The physician may also be elsewhere than in the operation room: a physician with a solid experience may instruct the person performing the anesthesia through a video conference connection, for example.

The arrangement may be applied to all anesthetical procedures, in which injected anesthetic is used.

Other applications of the arrangement in addition to epidural anesthesia include a plural number of anesthetical procedures, such as dentistry applications.

The arrangement may be utilized as an assisting positioning tool together with or instead of X-ray and other imaging techniques.

The arrangement may be used for educational purposes for example when medical students practice how to find the correct location for the anesthetic.

For example, it is possible to examine a nerve by using the arrangement to supply a current or voltage to a nerve tissue and to measure the response of the nerve tissue. On the basis of the values measured with the needle it is possible to deduce whether the nerve tissue subject to the measurement is healthy or damaged. Similarly, it is also possible to monitor the recovery of the functionality of a nerve tissue damaged in connection with the removal of a wisdom tooth, for example.

According to an embodiment the arrangement is used for monitoring the healing of a tissue by measuring the electrical and other values of the target to be monitored. The values may be compared with corresponding known values of a healthy tissue and thus the healing of the tissue after a bone fracture or surgery, for example, may be monitored. Likewise, it is possible to examine the degree of inflammation or recovery of a muscle or joint tissue without the burden of irradiation caused by X-ray equipment and the like.

According to another embodiment the arrangement is used for administering drugs. Examples that may be cited include the administering of an analgesic directly to the tissue in the area causing the pain and administering a colorant or radionuclides to a malignant tumour, for example, either for treatment or for purposes of imaging the area, which imaging may be carried out not only by visual observation but also by a gamma camera, for example, or some other detector. Further, the arrangement may be applied to administer botulin (neurotoxin) to an area with the aim to cause a muscle tissue to relax. Further still, the arrangement may be applied to administer substances meant for controlling growth factors and tissue growth, or tissue specialization in general, precisely to the desired tissue. In addition, the arrangement may be applied when administering antibiotics to targets out of reach for blood circulation, such as abscesses. If the target is inside a bone, the arrangement may be provided with a cutting blade and a mechanism that allow the needle to penetrate through the bone into the target. Likewise, when necessary, the arrangement may be provided with a vacuum pump etc. for removing suppuration from a lesion and the surrounding area. Further still, the arrangement may be applied in the above-mentioned targets also to biopsy.

An arrangement of the invention may also be applied to biopsies, for example, and to all measurements of electrical conductivity in a living tissue. The arrangement is applicable to taking a sample from spinal fluid, i.e. puncturing, which is an applicable method particularly when children are concerned. The arrangement is also suitable for taking a sample from an inflamed tissue or a malignant change. The electrical conductivity values of a healthy tissue differ from those of a sick one. The device enables taking a sample from an area as “representative” as possible. This avoids taking the biopsy by mistake from a healthy area in a partly sick organ or tissue. Technical solutions relating to biopsy or to the detaching of a sample and taking it out of the body are known per se and are therefore not going to be discussed in more detail in this context.

A sample may also be taken for examining the patient's response to specific drugs. It is possible to examine from the sample whether the patient's receptors, such as liver receptors, employed by the drug are working. In that case the system may be provided with an analysis unit.

The arrangement may also be used for a biopsy to determine, with separate means, the drug content of the target area and to verify whether a substance used in cytostatic treatment, for example, such as a treatment involving a radionuclide, finds its way to the desired area sufficiently well.

The arrangement also allows other targets to be positioned from which a sample may then be taken. An example such targets is a lymph node. In a patient suffering from breast cancer a sentinel lymph node is the first lymph node that the lymph and eventual cancer cells carried by it enter directly from the tumour. Cancer is in fact assumed to spread first to the sentinel lymph node. It is also assumed that if the sentinel lymph node in the armpit is healthy, the rest of the nodes are healthy, too, and the patient will not benefit from armpit evacuation, i.e. the emptying of the armpit, which means removing the tissue and the lymph nodes located in the armpit. There may be one or more sentinel nodes and they may be located also outside the armpit area.

Some arrangements of the invention may be applied in the case of the sentinel lymph node in such a way that, firstly, the system may be used for a biopsy from the sentinel lymph cell. The arrangement is well suited for doing the biopsy, because due to the abnormal electrical conductivity value in a malignant tissue, it allows the sample to be taken from a point of the lymph node which is as representative as possible. This may be implemented with gamma camera assistance or without a camera. Likewise, the arrangement may be used for injecting a radionuclide or cytostatic to a lymph node in need of treatment. The arrangement may also detect a sentinel lymph node containing malignant tissue on the basis of an intravenously introduced radionuclide, because due to the high radionuclide content the electrical conductivity of the node is different from that of the surrounding area.

Some embodiments of the invention may be used for removing a substance from a tissue, for example for removing cumulated fluid from tissues. In that case the system may be provided with a vacuum pump, for example, to ensure efficient removal of fluid from the tissue. The system may be provided with a feedback coupling based on electrical conductivity values, for example, so that fluid is only removed when there is an excess amount of it in the tissue.

The system is also applicable to stem cell treatments, for example for taking a sample of fat and then selecting a suitable tissue and the anatomical target that is to be the subject of the stem cell treatment. One example of such treatments that may be mentioned is the treatment of tendon damages.

It is also possible to use the arrangement as an intelligent drain or tube through which secretory products leave the body, the system removing accumulated fluids only when there is an excess amount of fluid in the tissue. If the inflammation secretion to be removed is thick, the diameter of the needle may be increased to a considerably larger size than the diameters of anesthesia needles normally used. Also in this application the arrangement may be provided with a vacuum pump which in its feedback coupling mode regulates its operation on the basis of the accumulated fluid and/or secretory product in the tissue. In that case the drain is in place for several days and may be connected to the arrangement a couple of times a day, for example. If on the basis of the electrical conductivity or other values of the tissue an excessive amount of fluid and/or inflammatory secretion in the tissue is detected, the vacuum pump becomes switched on and, when the task is completed, it is switched off. In a normal situation this procedure may be carried out by a nurse at an outpatient department, for example, and the intermediate periods the patient may spend at home.

The arrangement of the invention is applicable to cosmetic treatments or cosmetic surgery. An example of such applications is liposuction from tissues. The needle 2 belonging to the arrangement may be guided to automatically find its way to an area containing fat tissue. The needle may also be provided with a vacuum pump, or the like, which removes a suitable amount of fat tissue from the area on the basis of the electrical conductivity values of the tissues, for example, measuring the amount of fat around the needle 2. As an auxiliary device a tube or a suction hose may be provided for conveying the fat tissue through the needle 2 away from the tissue. When an appropriate amount of fat tissue is reached, the feedback coupling mechanism in the arrangement automatically terminates the suction of the fat tissue from the tissue. A desired final amount of fat tissue may be programmed in advance. The arrangement is particularly well suited for the removal of small fatty accumulations requiring precision. An example of this kind of procedure is the removal of fat tissue accumulating to the skin on an eyelid.

In cosmetic treatments the arrangement may also be applied to administering a substance to be injected under uneven or loose skin. In that case the patient's own lipocytes or a physiological salt solution is injected subcutaneously in an amount required to achieve a degree of skin elasticity possibly programmed in advance to the system. The system is capable of determining subcutaneous elasticity by means of a pressure sensor, for example, or by measuring the electrical values of tissues. The apparatus may be additionally provided with a feedback coupling mechanism which switches off the system and causes the injection of the substance into the tissue to stop, when a threshold value indicating suitable skin elasticity is reached.

The arrangement may also be used for smoothing out wrinkles and scars by means of hyaluronic acid, for example. A prerequisite in that case is that the acid can be subcutaneously injected precisely to the desired target. An example of such targets is facial mimic wrinkles.

The arrangement may be connected to a computer for visualizing the desired model or structure of the face or the body. The visualized model or structure allows the system to be controlled in such a way that the patient's face or body may be modified as desired. To produce the model on the patient's face, for example, thread lifting may be used. In that case the system may be used to determine a suitable number of barbed threads to support and lift the skin and the tissue and their insertion under the skin by minisurgery. This allows loose areas of the face and the neck, for example, to be lifted. Likewise, it is possible to melt cosmetically disturbing fat tissue under the jaw, for example, by injection therapy. In that case the needle 2 is automatically guided to the fat tissue into which substance dissolving fat (lipolytic substance) may be injected. The arrangement may be used also for monitoring the success of the treatment and the dissolving of the fat in the area.

A similar arrangement may be used for example in a disease called atherosclerosis for removing fatty accumulations (cholesterol) from veins by lipolytic substances. In that case the tissue information obtained by the arrangement guides the needle 2 to a vein containing cholesterol accumulation. Through the needle 2 a dissolving agent is injected into the accumulated fat. The arrangement may be provided with feedback coupling that switches off the injection when the cholesterol has dissolved.

Botulin affects at the points of connection between a nerve and a muscle and at the ends of autonomous nerves, causing a weakening in their activity. At cosmetically problematic locations the weakening of muscle activity produces the desired result, such as the smoothing of wrinkles in the skin. A similar treatment may be used to control or entirely stop a harmful teeth grinding inclination of a patient. In that case botulin is injected to the occlusion muscles to cause a partial numbness of the muscles. The system allows the degree of the numbness to be regulated and measured. Botulin is to be applied precisely to the desired area. The arrangement comprising the needle 2 is well suited for both determining the location of the target and for controlled administering of a small botulin dose to the target.

Botulin and similar substances may be used for treating patients suffering from neurodynia. The arrangement allows the substance to be injected with precision to the area of a possibly damaged nerve causing the neurodynia. The substance paralyses the action of the nerve, thereby relieving or stopping also the pain in the area.

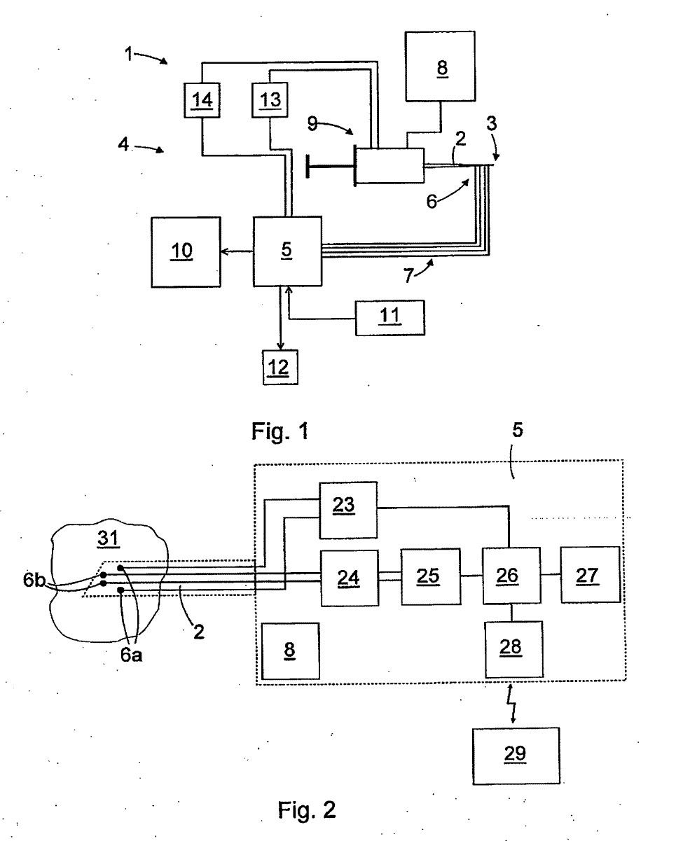

FIG. 2 is a schematic diagram of the processing unit 5 of the arrangement of the invention.

The processing unit 5 is connected to electrodes 6a, 6b by wires arranged to the needle 2. The processing unit 5 comprises a signal source 23, A/D converter 25 and microprocessor 26 and wiring or other electrical leads between them. Instead of wires, the system may employ wireless data transfer.

Through the wires the signal source 23 supplies a signal to two outermost electrodes 6a. The innermost electrodes 6b capture the signal coming from a tissue 31. The signal travels on the wires through a filter 24, if any, to the ND converter 25. Converted into a digital format, the signal continues to the microprocessor 26 for processing, from where the signal may be read. This may be assisted by a suitable user interface 27, known per se, which may comprise the signalling device 12 shown in FIG. 1, such as a component producing a sound signal or a visible signal or some other signal. The signal may also be transferred to a transmitter/receiver 28, which may in turn communicate with a PC or some other external user interface 29, which typically comprises the detection means 10 and the signalling means 12 shown in FIG. 1. In order to operate, the system requires a power source 8 that may be arranged to the processing unit 5 or externally to it, as shown in FIG. 1. All parts of the processing unit 5, except for the external user interface 29, are integrated into the anesthesia syringe. The arrangement 1 as a whole may be integrated into the syringe, catheter, sampling device or a similar device comprising a needle 2 to be monitored with the arrangement.

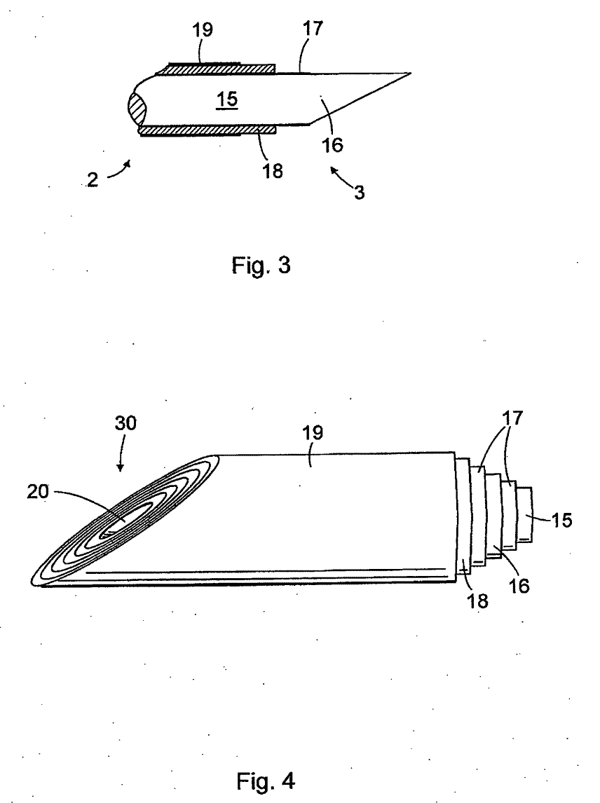

FIG. 3 is a schematic view of a needle belonging to the arrangement of the invention, and FIG. 4 is a schematic view of a second needle belonging to the arrangement of the invention. The Figures show only the distal end 3 of the needle.

The needle 2 of FIG. 3 is solid, i.e. its frame 15 does not have an elongated conduit. This kind of needle 2 may be used for example for identifying and examining a patient's tissues or for teaching the correct way to inject. Naturally the needle 2 may be hollow as well.

The needle 2 has two electrodes arranged thereto. A first electrode 16 is the frame 15 of the needle, which is made of a suitable electrically conductive material, typically metal, such as steel. The first electrode 16 is thus at the very outermost tip of the needle 2. The second electrode 18 is made of an electrically conductive material layer surrounding the needle frame 15 and insulated from it with a first insulation layer 17 arranged between the electrodes 16, 18. The first insulation layer 17 covers only part of the needle frame 15, leaving a small part of it exposed so that the first electrode 16 may come into contact with the tissue around it.

The material layer forming the second electrode 18 is coated with a second insulation layer 19 which forms a major part of the outer surface of the needle 2, leaving the material layer forming the second electrode 18 exposed so that it may come into contact with the surrounding tissue.

As already stated, the number of the electrodes may be two or more, because the electrode structure of FIG. 3 may be implemented with any number of electrodes.

The frame 15 of the needle of FIG. 4 has a longitudinal channel 20. The electrodes 16 and 18 are insulated from one another by insulation layers 17. The outermost layer is the second insulation layer 19. In the needle of FIG. 4 both electrodes 16 and 18 extend to the bevelling 30 at the needle tip, which distinguishes them from the needle of FIG. 3. The active surfaces of the electrodes 16 and 18, i.e. the electrode surfaces interacting with the tissue around the needle, are as close as possible to the outermost point 50 of the distal tip 3 of the needle, which is advantageous in most applications.

The needle 2 of FIGS. 3 and 4 may be made by coating the needle frame 15 alternately with an insulation material and an electrode material. The needle frame 15 may be made of stainless steel, for example. The frame is then coated with an insulating material. Materials coming into contact with the tissues must be biocompatible. The coating must also be tight and non-scaling. Suitable insulation materials are polymer coatings, for example propylene and Teflon, and some ceramic coatings. The insulation may be implemented for example by an immersion method, when propylene is used for instance, or by coating. Conceivable coating methods include a nano-coating method implemented by a sol gel technique, laser coating, plasma coating, vacuum coating and flame spray coating.

After having been produced onto the frame 15, the insulation layer is coated with metal. The above mentioned coating methods are suitable for the coating. Materials that may be used for the coating include gold, platinum or combination metals, such as platinum-iridium. Other possible materials are electrically conductive polymers and ceramic materials compatible with the tissue.

Alternating coating of the needle frame with the conductive substance and the insulation is continued until a desired amount of electrodes and, at the same time, conductors have been obtained. The size of the electrodes thus produced is modified by leaving an area of a varying size uncoated at the tip part of the needle on a next insulation round. The annular conductor part thus left at the needle tip may be either coated with insulation or left as a part of the electrode.

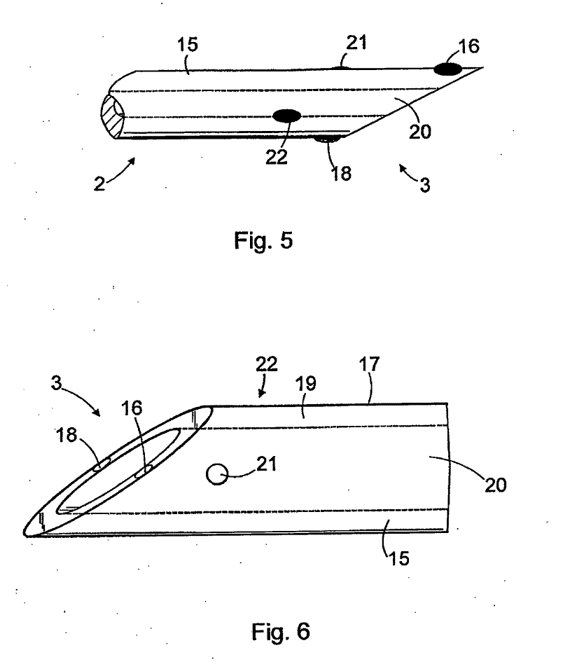

FIG. 5 is a schematic view of a third needle belonging to the arrangement of the invention. Also FIG. 5 only shows the distal end 3 of the needle.

The needle 2 is hollow, i.e. its frame 5 is provided with a conduit 20 in the longitudinal direction of the needle. The distal end 3 is provided with four electrodes: a first 16, second 18, third 21 and fourth electrode 22. All electrodes 16, 18, 21, 22 are arranged to the outer surface of the frame 15 and placed to the immediate vicinity of the bevel 30 at the distal tip 3 of the needle. Here the electrodes are round discrete pieces that are electrically isolated from one another and, when necessary, from the needle frame 15—the latter is not needed if the frame 15 is made of an electrically non-conductive material. When the material of the frame 15 is electrically conductive, it may be isolated from the electrodes 16, 18, 21, 22 by coating the frame 15 with a non-conductive coating and by arranging the electrodes on top of this, or, alternatively, with an electrode structure comprising an insulation material layer insulating the electrical parts of the electrode from the needle frame 15.

The first electrode 16 closest to the outermost point 50 of the distal tip 3 of the needle is preferably arranged at a distance of less than 1.0 mm, for example 0.9 mm, 0.8 mm or 0.7 mm, from the outermost point 50 in the longitudinal direction of the needle. The rest of the electrodes 18, 21 and 22 are preferably arranged at a distance of about 1 mm from the first electrode 16.

FIG. 6 is a schematic view of a fourth needle in the arrangement of the invention. The electrodes 16, 18 and 21, 22 are located in pairs symmetrically on both sides of the needle frame 15. The electrodes may also be placed at different distances from the outermost tip of the needle, in which case the distance between the electrodes slightly grows. An electrode has typically a round shape, although other geometries are also possible. The electrodes 16, 18 in the first electrode pair may be close to the needle tip 3 and the second electrode pair 21, 22 further away on the needle frame. The size of the electrodes is dependent on the thickness of the needle so that the electrodes are significantly smaller than the frame part of the needle. The greater the distance between the electrodes, the greater the tissue volume they measure. Generally, by selecting the number, location, such as distance, of the electrodes and the properties of the power supply and measurement electrodes it is possible to influence the reach of the measurement, i.e. how close or deep or where in the vicinity of the electrodes the conductivity of the tissue can be measured.

In the needle shown in FIG. 6 the electrodes 16, 18, 21, 22 have diameters which are significantly shorter than the needle frame. The first electrodes 16, 18 are located at the bevel 30 of the distal tip of the needle, where they are embedded. The electrodes are round and conform to the shape of the needle frame 15.

The second electrodes 21, 22 are preferably at less than 1 mm from the first electrodes 16, 18 on opposite sides of the needle frame 15 and at an equal distance from the outermost point 50 of the distal tip of the needle. In this embodiment the four electrodes 16, 18, 21, 22 are all equal in size. The second electrodes 21, 22 are embedded in the frame part of the needle. Otherwise the distal tip 3 of the needle is coated with an insulation layer 19.

It is naturally possible to use a needle 2 provided with electrodes implemented as in FIG. 3 or 4 and with electrodes of FIG. 5. Generally speaking, electrodes arranged to the needle 2 may either be all alike as to their size, shape and structure, or at least one electrode may be different from the rest.

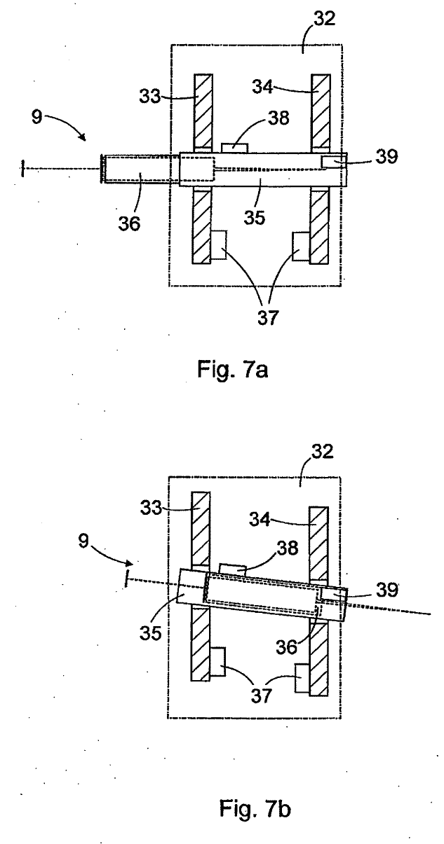

FIG. 7a is a schematic view of a master station of the arrangement of the invention in a first position, FIG. 7b showing the same station in a second position.

Sometimes it is problematic to find the correct injection spot for inserting a needle from the surface of the skin or mucosa into the tissue. Likewise, bringing the needle into the correct angle may cause problems. This is particularly difficult in a situation where the direction of the needle must be changed for example after the needle tip has come into bone contact within the tissue. In that case the needle must be re-directed a number of times during a single anesthesia procedure. Imprecision of the hand may also be a reason why a physician easily misses the target from above or passes by it and hits a wrong tissue.

If the needle tip hits a bone tissue by mistake, the distal tip of the needle may bend, i.e. become hooked, and break. In connection with a spinal cord anesthesia this may cause paraplegia, for example. If the needle unintentionally hits a nerve tissue, similar damage and/or permanent dysesthesia may occur.

The arrangement of the invention may comprise a master station 32 to be arranged to the patient's skin or mucosa, an embodiment of which is schematically shown in FIGS. 7a and 7b. The master station contains a frame structure attachable to the patient, his/her bed—if the patient is in narcosis, for example—to the frame of an imaging device or a wheelchair or the like. The frame structure has an upper plate 33 connected thereto and, at a distance from it, a lower plate 34 arranged in a parallel direction. The plates 33, 34 have openings extending through them. The location of the plates 33, 34 in relation to one another and, at the same time, to the frame structure 32 may be changed two-dimensionally in the planar direction of the plates. The frame structure further comprises a guide tube 35 turnably attached to the openings in the plates 33, 34 so that the longitudinal axis of the guide tube 35 pierces the openings. Inside the guide tube 35 there is arranged a fastening tube 36 that may slide in the guide tube 35 in the direction of the longitudinal axis thereof. The guide tube 35 and the fastening tube 36 thus form a telescopic structure.

The master station 32 comprises at least one and in this case two power members 37 connected to the plates 33, 34 and arranged to move the plates 33, 34 in two dimensions in the direction of their plane. Further, the master station 32 has a second power member 38 arranged to adjust the length of the telescopic structure formed by the guide tube 35 and the fastening tube 36. The power member 37 and the second power member 38 is a servo, for example, or the like.

The syringe 9 or some other already disclosed device comprising a needle is fastened to the fastening tube 36 by suitable fastening means. The fastening of the needle may be ensured for example by reducing the diameter of the fastening tube 36 by means of a clamping tool.

Now the position of the needle may be changed by moving the plates 33, 34 and the length of the telescopic structure. In FIG. 7b the upper plate 33 has been moved upward in the direction of the figure, whereby the needle has tilted to point below the horizontal level. The fastening tube 36 has been moved inside the guide tube 35, whereby the needle has moved forward and out of the guide tube 35. The position of the needle is changed by means of a control unit controlling the power members 37 and 38. The control unit may be a control card comprising a microprocessor, for example, that is capable of collecting the necessary information through wireless data transfer, for example, either from a sounding means 39—to be discussed below—or from the information obtained through the electrodes of the needle. The control unit is known per se and therefore is not going to be discussed in more detail in this specification, neither is it presented in the figures.

The master station shown in FIGS. 7a, 7b further comprises sounding means 39, which are arranged to detect the tissue behind a patient's skin or mucosa. The sounding means 39 may comprise for example an ultrasound transmitter and receiver known per se. The sounding means are preferably connected to the processing unit 5 already mentioned.

The master station 32 may comprise a base that attaches easily to the skin, for example, and the material of which may be silicon, or the like, in which case the frame structure is arranged onto the base. The frame structure may also “float”, so to speak, on the base. In that case the base contains silicon gel, for example, or similar material. The attachment of the master station 32 may be ensured by means of a suitable adhesive, for example, or by a suction pad making use of a vacuum.

It is evident that the master station 32 may be implemented in various ways. Instead of two plates 33, 34 it is possible to use one plate, for example, and the needle may be moved by means of a mechanical arm or the like.

The master station 32 is fastened close to an area from which the needle is meant to be taken into the tissue. The sounding means 39 identify bone tissue, such as a joint for example, already from the surface of the skin or mucosa. In an epidural anesthesia administered to the back, for example, the needle is directed between two spinal vertebrae.

When the needle has reached the intended injection spot, also the lower plate 34 becomes attached through its own mechanism to the surface of the skin. This ensures that it does not become detached from the patient when the needle starts to proceed in the tissue. The mechanism may be for example a suction pad, a mechanical catch or the like. The angle of the needle is then changed with the above power members 37 to suit the target. A suitable needle angle prevents the needle from hitting bone tissue, for example, on its path. The arrangement 1 may use a signal light or sound signal, for example, to inform that the needle is in the correct position with regard to the tissue or tissue part aimed at.

The movement of the advancing needle and its location in the tissue is monitored as described with reference to FIGS. 1 and 2.

The arrangement may further comprise automated means for guiding the distal tip of the needle to the tissue or tissue part aimed at. The needle may be automatically directed to a new route where it bypasses the bone tissue, for example. If the route needs to be changed, the master station may retract the needle slightly to facilitate its progress in the new direction.

Before injecting the anesthetic, the physician may receive information on the correct position of the needle tip with a signal light or sound signal, for example. In the last step the anesthetic is injected from a vial or other container through the needle to the target manually or by means of a motor or the like.

The master station provides a number of advantages. Firstly, it facilitates the injecting of the needle to the correct location and at a right angle to the skin or the mucosa. Also when the needle is injected into the tissue, needle movements assisted by a motor are significantly more precise than hand movements. With the motor the dosage of the anesthetic is more accurate and precise than manual injection. After the master station has been attached the system may preferably act independently, whereby the anesthetic procedure as a whole takes place at the press of a button.

The master station may be utilized for example for taking a sample from spinal fluid, i.e. for puncturing, which is a useful method with children in particular.

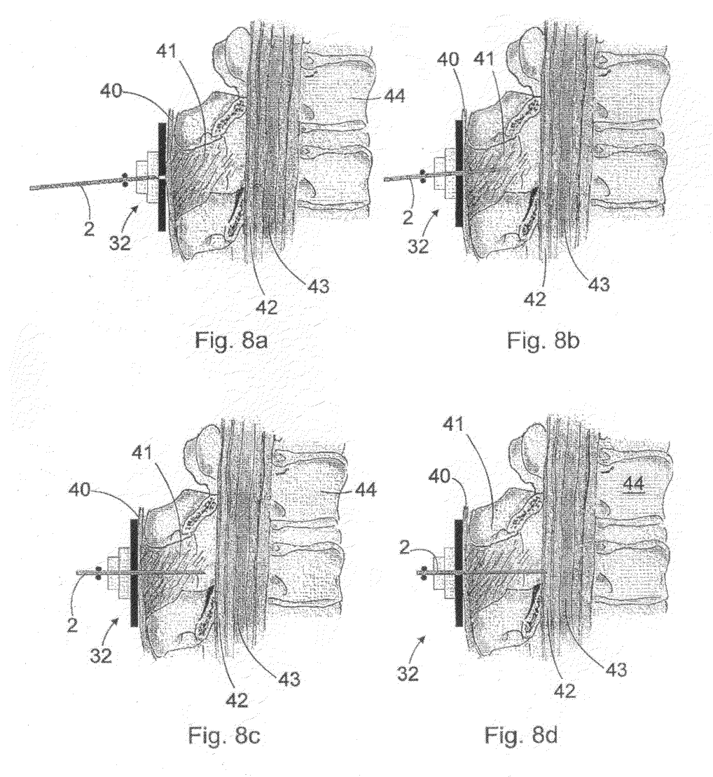

FIGS. 8a to 8d are schematic views of an arrangement of the invention in connection with epidural anesthesia. In FIG. 8a the needle 2 is placed to the master station 32. The master station searches for the correct location for the anesthetic and the correct angle for the needle 2 by means of sounding devices and moves the needle with the power members already mentioned to a suitable location on the surface of the skin 40.

In FIG. 8b the master station 32 has moved the needle 2 through the muscular tissue 41 towards the point of anesthesia aimed at, i.e. the epidural space 42. The angle of the needle 2 is chosen so that it does not hit the bone tissue of the spinal vertebrae 44 on its route.

In FIG. 8c the master station 32 re-directs the needle 2 and moves it forward.

In FIG. 8d the distal end of the needle 2 is in the epidural space 42 where the master station 32 injects the anesthetic.

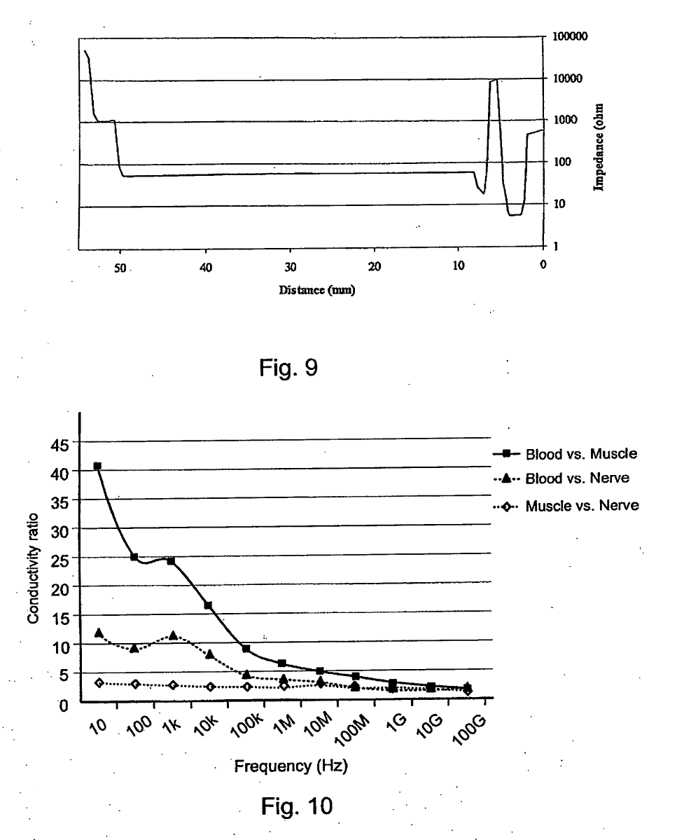

FIG. 9 shows a typical bioimpedance curve modelled with the arrangement of the invention. It is easy to follow from the curve how the needle proceeds in the epidural and/or spinal space during anesthesia of the back area. The first high point at the early part of the curve is caused by the skin. The highest peak at the final part of the curve represents the arrival of the needle at the epidural space, where electrical conductivity is extremely poor. Two pits in the final part are caused by periosteum and the spinal space, whose electrical conductivity values are high.

FIG. 10 shows the conductivity ratio of some tissue types as a function of the measurement frequency. Bioimpedance drops in most tissues as the measurement frequency increases. It is possible to deduce from FIG. 10 that the measurement frequency is significant when different tissue types are to be distinguished from one another. For example, if blood tissue is to be distinguished from nerve tissue, it is most advantageous to use as low measurement frequency as possible, because these tissues become rapidly indistinguishable from one another as the measurement frequency increases. Muscle tissue becomes less distinguishable from nerve tissue when the measurement frequency exceeds one kilohertz. Then again, whether a higher or a lower frequency is used for distinguishing a blood tissue from a muscle tissue does not make much difference.

The measurement frequency used in bioimpedance measurement is preferably selected according to the tissues where the distinction is to be made. For example, in a measurement made in connection with spinal cord anesthesia, it is primarily the epidural space that is to be distinguished from the spinal space, whereas in a measurement made in connection with nerve block anesthesia of the lower jaw a nerve and a vein tissue must be distinguished from muscle tissue. The measurement arrangement is preferably provided with means that enable bioimpedance measurements to be carried out at different measurement frequencies, or with a signal generator capable of producing simultaneous electric currents of different frequencies, the currents being supplied through electrodes to the tissue in one and the same measurement event.

It should be noted that the inventors surprisingly observed that the phase angle of bioimpedance reacts particularly sensitively to a change in the tissue type. Hence it is most advantageous to determine not only the magnitude of the bioimpedance but also its phase angle. The values of the phase angle of a fat tissue, for example, are small, about 3°, whereas the phase angle of a dry skin is of the order of 80°, that of a muscle tissue about 30° and a blood tissue about 20°.

In some cases the features of this application may be used as such, irrespective of the other features. However, the features disclosed here may be combined, when necessary, to produce different combinations.

The drawings and the related specification are only intended to illustrate the invention. The details of the invention may vary within the scope of the claims.

1-23. (canceled)

24. An arrangement for determining the position of a needle in an organ system, the arrangement comprising
a needle having a distal tip;
a measurement circuit measuring the bioimpedance of tissues;
a detection means, the detection means being connected to the measurement circuit measuring the bioimpedance and arranged to detect the progress of the distal tip of the needle in the tissue on a real-time basis from the bioimpedance measured from the tissues,
the arrangement further comprising at least two electrodes arranged to the distal tip of the needle, the electrodes being coupled to the measurement circuit; wherein
all electrodes measuring bioimpedance and needed for monitoring the progress of the distal tip of the needle are arranged to the distal tip of the needle.

25. An arrangement according to claim 24, wherein the number of the electrodes is two.

26. An arrangement according to claim 24, wherein the number of the electrodes is four.

27. An arrangement according to claim 24, wherein one of the electrodes is the needle frame, which is electrically insulated from the rest of the electrodes arranged to the needle.

28. An arrangement according to claim 24, wherein at least one electrode is made of an electrically conductive material layer which surrounds the needle frame and which is electrically insulated from the rest of the electrodes arranged to the needle.

29. An arrangement according to claim 24, wherein at least one electrode is a discrete piece formed to the outer surface of the needle.

30. An arrangement according to claim 24, wherein that the detection means is a display device.

31. An arrangement according to claim 24, comprising means for identifying the force that resists the progress of the needle in the tissue.

32. An arrangement according to claim 31, wherein the means comprise a pressure sensor and a power sensor.

33. An arrangement according to claim 24, wherein the needle is hollow and comprises a conduit for supplying fluid substances into and/or out of the body.

34. An arrangement according to claim 33, comprising a container connected to the needle conduit.

35. An arrangement according to claim 24, comprising a master station to be arranged to the patient's skin or mucosa, the master station including
a frame structure attachable to the patient and
at least one power member arranged to move the needle in relation to the frame structure.

36. An arrangement according to claim 35, wherein the master station comprises sounding devices arranged to identify subcutaneous tissue through the patient's skin.

37. An arrangement according to claim 36, wherein the sounding devices comprise an ultrasound transmitter and receiver.

38. An arrangement according to claim 24, comprising memory means for storing an anatomic model created on the basis of anatomical data and means for adjusting the bioimpedance information measured by the electrodes to the model in such a way that the position of the distal tip of the needle in relation to the anatomic model is reproducible in the detection means.

39. An arrangement according to claim 38, wherein the anatomical data comprise generally known information on species-specific anatomy.

40. An arrangement according to claim 38, wherein the anatomical data comprise material measured from the patient's own anatomy.

41. An arrangement according to claim 24, comprising a signalling device arranged to produce a discernible sign when the distal tip of the needle is in a specific tissue.

42. An arrangement according to claim 41, wherein the discernible sign is a visually discernible sign.

43. An arrangement according to claim 41, wherein the discernible sign is a sound signal.

44. An arrangement according to claim 24, wherein the electrode closest to the outermost point of the distal tip of the needle is arranged at a distance less than 1.0 mm from the outermost point, when seen in the longitudinal direction of the needle.

45. An arrangement according to claim 44, wherein the rest of the electrodes are arranged at a distance of about 1 mm at the most from the electrode closest to the outermost point 50 of the distal tip, when seen in the longitudinal direction of the needle.

46. A method in cosmetic treatment or cosmetic surgery, the method comprising the steps of
arranging for use a needle with a distal tip, in which needle all electrodes measuring bioimpedance and needed for monitoring the progress of the distal tip of the needle are arranged to the distal tip;
arranging for use a measurement circuit measuring the bioimpedance of tissues, and a detection means that is connected to the measurement circuit measuring bioimpedance;
arranging the detection means to detect the progress of the distal tip of the needle in the tissue on a real-time basis from the bioimpedance measured from the tissues;
connecting the electrodes to the measurement circuit;
inserting the needle into the patient's tissue; and
measuring the bioimpedance of the tissue.
